(19) United States
(12) Patent Application Publication
(10) Pub. No.: US 2016/0152539 A1
(43) Pub. Date: Jun. 2, 2016

(54) PHOSPHORAMIDITE DERIVATIVES IN THE HYDROFORMYLATION OF UNSATURATED COMPOUNDS

(71) Applicant: EVONIK DEGUSSA GMBH, Essen (DE)

(72) Inventors: Katrin Marie DYBALLA, Recklinghausen (DE); Robert FRANKE, Marl (DE); Dirk FRIDAG, Haltern am See (DE); Eduard BENETSKIY, Ryazan (RU); Armin BÖRNER, Rostock (DE); Detlef SELENT, Rostock (DE)

(73) Assignee: EVONIK DEGUSSA GMBH, Essen (DE)

(21) Appl. No.: 14/906,644

(22) PCT Filed: Jul. 22, 2014
(86) PCT No.: PCT/EP2014/065733

§ 371 Date: Jan. 21, 2016

(30) Foreign Application Priority Data
Jul. 23, 2013 (DE) 102013214382.6

(51) Int. Cl. C07C 45/50; B01J 31/18; C07B 41/06; C07F 15/00; C07F 9/6571

(52) U.S. Cl. CPC C07C 45/50; C07F 15/0073; C07F 9/657154; C07B 41/06; B01J 31/186; B01J 2231/321; B01J 2531/822

(57) ABSTRACT

The invention relates to: a) phosphoramidites of formula (I), wherein Q is selected from substituted or unsubstituted 1,1′-biphenyl groups, R¹ stands for hydrogen, and R² stands for C₁-C₁₀ alkyl groups, substituted or unsubstituted C₄-C₆ cycloalkyl groups, or phenyl groups and wherein R² is not a tertiary butyl group; b) transition-metal-containing compounds of the formula Me(acac)(CO)L, wherein Me=transition metal and L of the general formula (II): wherein Q is selected from substituted or unsubstituted 1,1′-biphenyl groups, R¹ stands for hydrogen, R² stands for C₁-C₁₀ alkyl groups, substituted or unsubstituted C₄-C₆ cycloalkyl groups, or phenyl groups, and R² is not a tertiary butyl group and wherein the transition metal Me is selected from ruthenium, cobalt, rhodium, and iridium; c) catalytically active compositions in the hydroformylation, which comprise the compounds mentioned under a) and b); d) method for the hydroformylation of unsaturated compounds by using the catalytically active composition mentioned under c), and e) multi-phase reaction mixture, containing unsaturated compounds, gas mixture, which comprises carbon monoxide and hydrogen, aldehydes, and the catalytically active composition described under c).

In terms of volume, hydroformylation is one of the most important homogeneous catalyses on the industrial scale. The aldehydes obtained thereby are important intermediates or end products in the chemical industry (Rhodium Catalyzed Hydroformylation, P. W. N. M. van Leeuwen, C. Claver, eds.; Kluver Academic Publishers: Dordrecht Netherlands; 2000. R. Franke, D. Selent, A. Börner, Chem. Rev. 2012, 112, 5675.). Hydroformylation with Rh catalysts is of particular significance.

For control of activity and regioselectivity of the catalyst, usually compounds of trivalent phosphorus are used as organic ligands. Particularly phosphites, i.e. compounds containing three P—O bonds, have become very widely used for this purpose (EP 0054986; EP 0697391; EP 213639; EP 214622; U.S. Pat. No. 4,769,498; DE 10031493; DE 102006058682; WO 2008124468).

Phosphoramidites, i.e. compounds having one or more P—N bonds rather than the P—O bonds, have to date been used only rarely as ligands in hydroformylation.

Van Leeuwen and coworkers (A. van Rooy, D. Burgers, P. C. J. Kamer, P. W. N. M. van Leeuwen, Recl. Tray. Chim. Pays-Bas 1996, 115, 492) were the first to study monodentate phosphoramidites in hydroformylation. Overall, only moderate catalytic properties were observed at the high to extremely high ligand/rhodium ratios of up to 1000:1. At the lowest ligand/rhodium ratio, or P/Rh ratio, of 10:1, a high isomerization activity and the formation of non-hydroformylated internal olefins was found. Only increasing the P/Rh ratio increased the TOF to a moderate 910 h⁻¹ and enhanced the selectivity.

The use of chiral phosphoramidites for asymmetric catalyses was claimed in WO 2007/031065, without giving working examples specifically for asymmetric hydroformylation. Chiral bidentate ligands each having a phosphoramidite unit have been used in various forms in asymmetric hydroformylation (J. Mazuela, O. Pàmies, M. Diéguez, L. Palais, S. Rosset, A. Alexakis, Tetrahedron: Asymmetry 2010, 21, 2153-2157; Y. Yan, X. Zhang, J. Am. Chem. Soc. 2006, 128, 7198-7202; Z. Hua, V. C. Vassar, H. Choi, I. Ojima, PNAS 2004, 13, 5411-5416).

Of paramount importance for the efficacy of the catalyst is the stability of the ligand towards various chemical agents before, during and after the catalysis (the latter in the case of intentional recycling). One of the main causes of the breakdown of phosphite ligands, which, unlike phosphines, are very stable towards oxygen, is the reaction with water, which leads to cleavage of the P—O bonds (Homogeneous Catalysts, Activity-Stability-Deactivation, P. W. N. M. van Leeuwen, J. C. Chadwick, eds.; Wiley-VCH, 2011, p. 23 ff.). The hydrolysis gives rise particularly to pentavalent phosphorus compounds which have lost most of their ligand properties. Water forms almost unavoidably under almost all hydroformylation conditions through aldol condensation of the product aldehydes.

In general, a greater tendency to react with nucleophiles is attributed to phosphoramidites than phosphites. This property is utilized widely, for example, for the synthesis of phosphites from phosphoramidites (e-EROS Encyclopedia of Reagents for Organic Synthesis. doi:10.1002/047084289X.rn00312; R. Hulst, N. K. de Vries, B. L. Feringa, Tetrahedron: Asymmetry 1994, 5, 699-708), but at the same time raises particular questions about the suitability thereof as ligands of long-term stability for catalysis. For example, the use of phosphoramidites as stabilizers for polyolefins is known, as disclosed by EP 0005500 A1.

The use of suitable phosphorus substituents can contribute to stabilization of phosphorus compounds at risk of hydrolysis. The only method described to date in the context of phosphoramidite ligands is the use of N-pyrrolyl radicals on the phosphorus (WO 02/083695). Substituents on the heterocycle, for example 2-ethylpyrrolyl (WO 03018192, DE 102005061642) or indolyl (WO 03/018192), improve hydrolysis stability still further.

The hydrolytic breakdown of phosphoramidite ligands can also be slowed by the addition of amines to the hydroformylation reaction, as taught in EP 1677911, US 2006/0224000 and U.S. Pat. No. 8,110,709.

However, the use of hydrolysis-stable pyrrolylphosphines or the addition of basic stabilizers greatly narrows the scope of application of the hydroformylation reaction to these working examples.

It is an object of the present invention to provide ligands for catalytically active compositions for chemical synthesis of organic compounds, especially the hydroformylation, the hydrocyanation and the hydrogenation of unsaturated compounds, which have a maximum or improved catalytic efficacy, measured as activity k_{obs}. [min⁻¹], even at significantly lower phosphorus/rhodium concentration ratios—P/Rh for short—compared to the ligands previously described in the prior art. This is especially relevant for use in industrial scale processes, since a higher activity—for example in the hydroformylation—means shorter residence times for the target products—aldehydes—in the reaction zone. Thus, yield-reducing further reactions of the target products, e.g. aldolization reactions, are reduced and the overall economic viability of the industrial process is optimized. As well as the ease of synthesis of the phosphoramidites, a high yield of product is to be achieved.

It has been found that, surprisingly, this object is achieved by use of phosphoramidites of the formula (I):

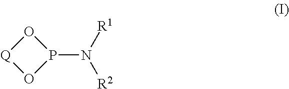

where Q is selected from substituted or unsubstituted 1,1′-biphenyl radicals, R¹ is hydrogen and R² is C₁-C₁₀-alkyl, preferably C₁-C₅-alkyl, or substituted or unsubstituted C₄-C₆-cycloalkyl or phenyl radicals and where no R² is a tert-butyl radical.

The present invention therefore provides phosphoramidites of the formula (I) as described above and in the claims.

Preferred substituted 1,1′-biphenyl radicals are those having, in the 3,3′ and/or 5,5′ positions of the 1,1′-biphenyl-2,2′-diol base skeleton, an alkyl radical, preferably a C₁-C₄-alkyl radical and more preferably a tert-butyl radical (t-Bu).

Preferred radicals for R² are unbranched and/or branched C₁-C₅-alkyl, substituted or unsubstituted C₄-C₆-cycloalkyl radicals, phenyl radicals excluding tert-butyl (t-Bu).

It may be advantageous when the phosphoramidites of the formula (I) are selected from those of the following formulae:

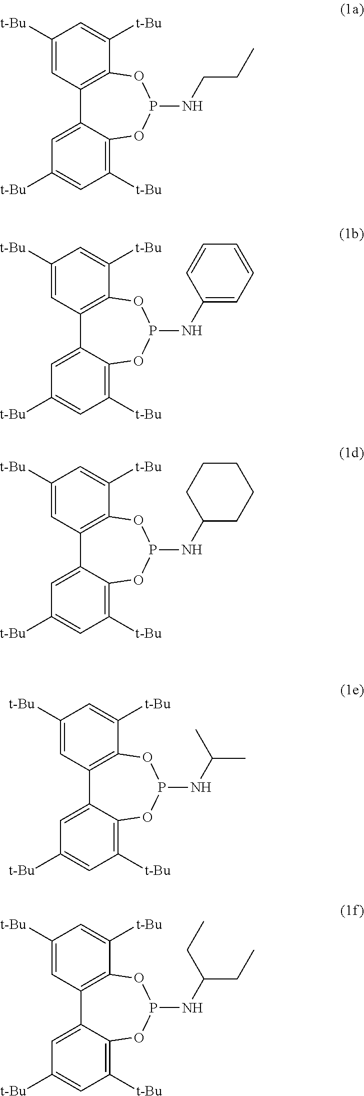 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1 ; CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

The present invention further provides transition metal compounds of the formula Me(acac)(CO)L with Me=transition metal and L of the general formula (I):

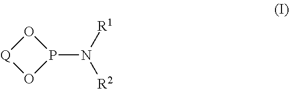

where Q is selected from substituted or unsubstituted 1,1′-biphenyl radicals, R¹ is hydrogen and R² is C₁-C₁₀-alkyl, preferably C₁-C₅-alkyl, or substituted or unsubstituted C₄-C₆-cycloalkyl or phenyl radicals, where R² is not a tert-butyl radical, and where the transition metal Me is selected from ruthenium, cobalt, rhodium and iridium, particular preference being given to rhodium.

Preferred substituted 1,1′-biphenyl radicals are those having, in the 3,3′ and/or 5,5′ positions of the 1,1′-biphenyl-2,2′-diol base skeleton, an alkyl radical, preferably a C₁-C₄-alkyl radical and more preferably a tert-butyl radical (t-Bu).

Preferred radicals for R² are unbranched and/or branched C₁-C₅-alkyl, substituted or unsubstituted C₄-C₆-cycloalkyl radicals, phenyl radicals excluding tert-butyl (t-Bu).

In preferred transition metal compounds of the formula Me(acac)(CO)L, L is selected from:

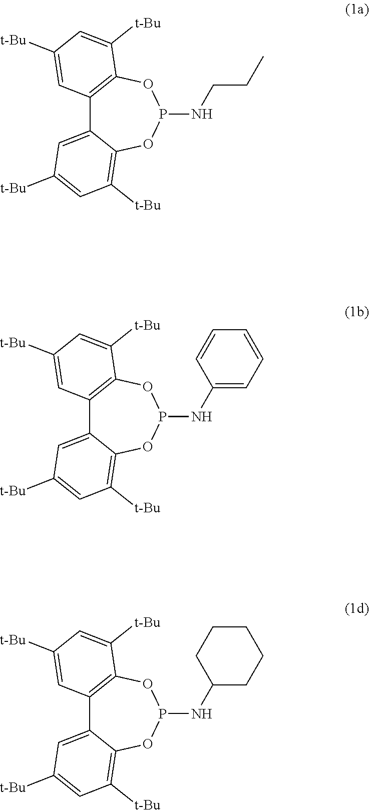 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1]

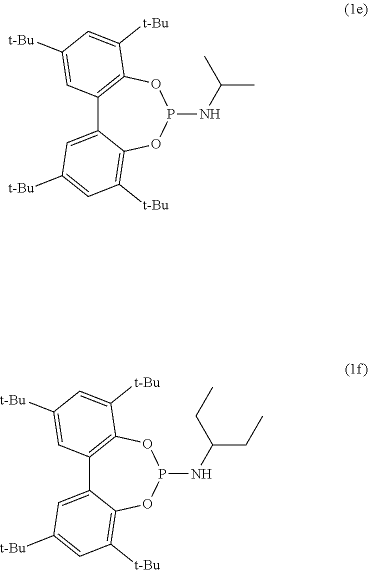 [SMILES: CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

Particularly preferred transition metal compounds of the formula Me(acac)(CO)L are those in which Me=rhodium and L is selected from

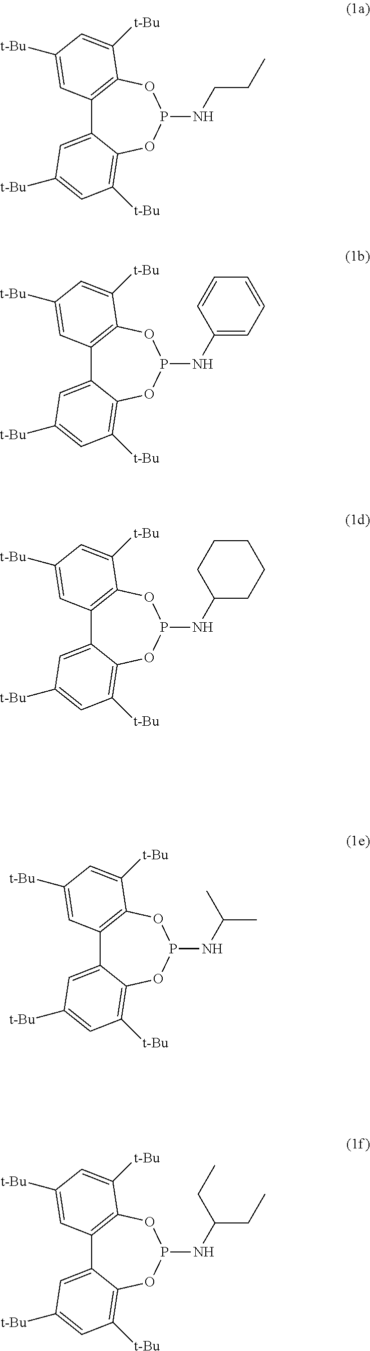 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1 ; CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

The transition metal can be contacted with the phosphoramidites as a precursor in the form of its salts, for example the halides, carboxylates—e.g. acetates—or commercially available complexes, for example acetylacetonates, carbonyls, cyclopolyenes—e.g. 1,5-cyclooctadiene—or else mixed forms thereof, for example Rh(acac)(CO)₂ with acac=acetylacetonate anion, Rh(acac)(COD) with COD=1,5-cyclooctadiene, and this reaction can be effected in a preceding reaction or else in the presence of a hydrogen- and carbon monoxide-containing gas mixture.

The present invention also provides catalytically active compositions in the hydroformylation comprising:

a) transition metal compounds of the formula Me(acac)(CO)L with Me=transition metal and L of the general formula (II):

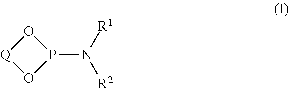

where Q is selected from substituted or unsubstituted 1,1′-biphenyl radicals, R¹ is hydrogen and R² is C₁-C₁₀-alkyl, preferably C₁-C₅-alkyl, or substituted or unsubstituted C₄-C₆-cycloalkyl or phenyl radicals, where R² is not a tert-butyl radical, and where the transition metal Me is selected from ruthenium, cobalt, rhodium and iridium, and is preferably rhodium.

Preferred substituted 1,1′-biphenyl radicals are those having, in the 3,3′ and/or 5,5′ positions of the 1,1′-biphenyl-2,2′-diol base skeleton, an alkyl radical, preferably a C₁-C₄-alkyl radical and more preferably a tert-butyl radical (t-Bu);

preferred radicals for R² are unbranched and/or branched C₁-C₅-alkyl, substituted or unsubstituted C₄-C₆-cycloalkyl radicals, phenyl radicals excluding tert-butyl (t-Bu);
preferably, L in the transition metal compound of the formula Me(acac)(CO)L is selected from:

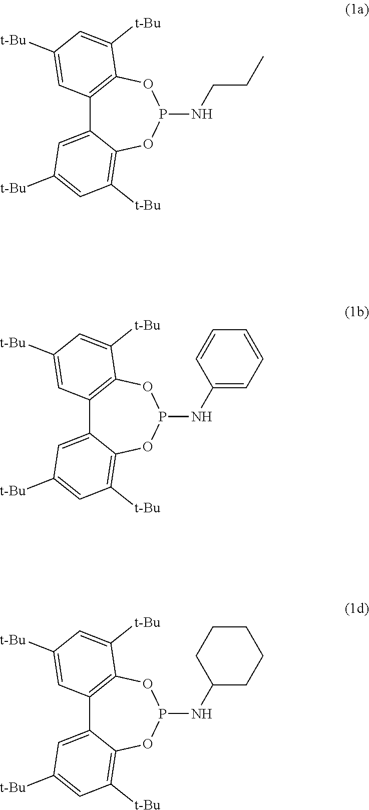 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1]

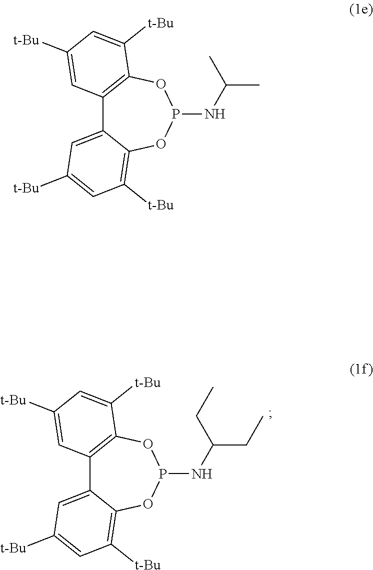 [SMILES: CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

b) free ligands of the formula (I):

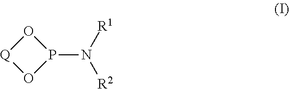

where Q is selected from substituted or unsubstituted 1,1′-biphenyl radicals, R¹ is hydrogen and R² is C₁-C₁₀-alkyl, preferably C₁—C-alkyl, or substituted or unsubstituted C₄-C₆-cycloalkyl or phenyl radicals, where R² is not a tert-butyl radical.

Preferred substituted 1,1′-biphenyl radicals are those having, in the 3,3′ and/or 5,5′ positions of the 1,1′-biphenyl-2,2′-diol base skeleton, an alkyl radical, preferably a C₁-C₄-alkyl radical and more preferably a tert-butyl radical (t-Bu);

preferred radicals for R² are unbranched and/or branched C₁-C₅-alkyl, substituted or unsubstituted C₄-C₆-cycloalkyl radicals, phenyl radicals excluding tert-butyl (t-Bu); preferred free ligands of the formula (I) are selected from:

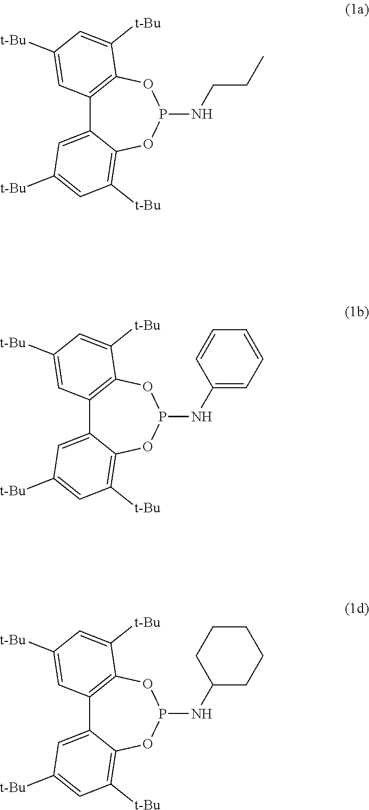 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1]

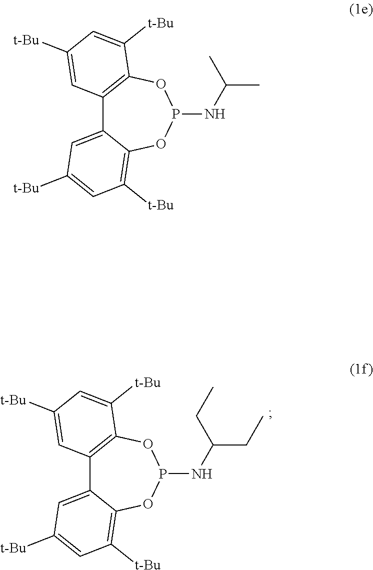 [SMILES: CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

c) solvents,

In the context of the present invention, solvents are regarded as being not only substances that have no inhibiting effect on product formation—having been added externally to the reaction mixture or initially charged therein—but also mixtures of compounds which form from side reactions or further reactions of the products in situ; for example what are called high boilers which form from the aldol condensation, the acetalization of the primary aldehyde product or else esterification, and lead to the corresponding aldol products, formates, acetals and ethers. Solvents initially charged externally in the reaction mixture may be aromatics, for example toluene-rich aromatics mixtures, or alkanes or mixtures of alkanes.

In general, high boilers are understood to mean those substances or else substance mixtures that boil at a higher temperature than the primary aldehyde products and have higher molar masses than the primary aldehyde products.

The present invention further provides:

for the use of the above-described catalytically active compositions in a process for hydroformylating unsaturated compounds and a process for hydroformylating unsaturated compounds using said catalytically active composition, where the unsaturated compounds are preferably selected from:
hydrocarbon mixtures from steamcracking plants;
hydrocarbon mixtures from catalytically operated cracking plants;
hydrocarbon mixtures from oligomerization processes;
hydrocarbon mixtures comprising polyunsaturated compounds;
olefin-containing mixtures comprising olefins having up to 30 carbon atoms;
unsaturated carboxylic acid derivatives.

The unsaturated compounds which are hydroformylated in the process according to the invention preferably include hydrocarbon mixtures obtained in petrochemical processing plants. Examples of these may include what are called C₄ cuts. Typical compositions of C₄ cuts from which preferably the majority of the polyunsaturated hydrocarbons has been removed and which can be used in the process according to the invention are listed in Table 1 below (see DE 10 2008 002188).

TABLE 1

| | | | | | Catalytic | |
|---|---|---|---|---|---|---|
| | Steamcracking plant | | Steamcracking plant | | cracking plant | |
| Component | HCC₄ | HCC₄/SHP | Raff. I | Raff. I/SHP | CC₄ | CC₄/SHP |
| isobutane | 1-4.5 | 1-4.5 | 1.5-8 | 1.5-8 | 37 | 37 |
| [% by mass] | | | | | | |
| n-butane | 5-8 | 5-8 | 6-15 | 6-15 | 13 | 13 |
| [% by mass] | | | | | | |
| E-2-butene | 18-21 | 18-21 | 7-10 | 7-10 | 12 | 12 |
| [% by mass] | | | | | | |
| 1-butene | 35-45 | 35-45 | 15-35 | 15-35 | 12 | 12 |
| [% by mass] | | | | | | |
| isobutane | 22-28 | 22-28 | 33-50 | 33-50 | 15 | 15 |
| [% by mass] | | | | | | |
| Z-2-butene | 5-9 | 5-9 | 4-8 | 4-8 | 11 | 11 |
| [% by mass] | | | | | | |
| 1,3 butadiene | 500-8000 | 0-50 | 50-8000 | 0-50 | <10000 | 0-50 |
| [ppm by mass] | | | | | | |

### Key:

HCC₄: typical of a C₄ mixture which is obtained from the C₄ cut from a steamcracking plant (high severity) after the hydrogenation of the 1,3-butadiene without additional moderation of the catalyst.
HCC₄/SHP: HCC₄ composition in which residues of 1,3-butadiene have been reduced further in a selective hydrogenation process/SHP.
Raff. I (raffinate I): typical of a C₄ mixture which is obtained from the C₄ cut from a steamcracking plant (high severity) after the removal of the 1,3-butadiene, for example by an NMP extractive rectification.
Raff. II/SHP: raft. I composition in which residues of 1,3-butadiene have been reduced further in a selective hydrogenation process/SHP.
SCC₄: typical composition of a C₄ cut which is obtained from a catalytic cracking plant.
CC₄/SHP: composition of a C₄ cut in which residues of 1,3-butadiene have been reduced further in a selective hydrogenation process/SHP.

Likewise usable in the process of the invention are unsaturated compounds or a mixture thereof selected from:
hydrocarbon mixtures from steamcracking plants;
hydrocarbon mixtures from catalytically operated cracking plants, for example FCC cracking plants;
hydrocarbon mixtures from oligomerization processes in the homogeneous phase and heterogeneous phases, for example the OCTOL, DIMERSOL, Fischer-Tropsch, Polygas, CatPoly, InAlk, Polynaphtha, Selectopol, MOGD, COD, EMOGAS, NExOCTANE or SHOP process;
hydrocarbon mixtures comprising polyunsaturated compounds;
unsaturated carboxylic acid derivatives.

Preferably, the unsaturated compounds or mixtures thereof used in the process according to the invention include unsaturated compounds having 2 to 30 carbon atoms, more preferably having 2 to 8 carbon atoms.

If polyunsaturated hydrocarbons or mixtures comprising them are used in the process according to the invention, the polyunsaturated hydrocarbons are preferably butadienes.

If unsaturated carboxylic acid derivatives are used in the process according to the invention as unsaturated compounds which are hydroformylated in the process according to the invention, these unsaturated carboxylic acid derivatives are preferably selected from fatty acid esters, more preferably from those fatty acid esters based on renewable raw materials. In the context of the present invention, renewable raw materials, as opposed to petrochemical raw materials based on fossil resources, for example mineral oil or hard coal, are understood to mean those raw materials which arise or are produced on the basis of biomass. The terms “biomass”, “bio-based” or “based on”, or “produced from renewable raw materials”, include all materials of biological origin which originate from what is called the “short-term carbon cycle”, and are thus not part of geological formations or fossil strata. More particularly, “based on renewable raw materials” and “on the basis of renewable raw materials” are understood to mean that, by the ASTM D6866-08 method (¹⁴C method), the appropriate proportion of ¹⁴C isotopes can be detected in the hydroformylation mixture of the fatty acid esters.

The identification and quantification of renewable raw materials can be effected to ASTM Method D6866. One characterizing feature of renewable raw materials is the proportion therein of the ¹⁴C carbon isotope as against petrochemical raw materials. With the aid of the radiocarbon method, it is possible to determine the proportion of ¹⁴C isotopes and hence also the proportion of molecules based on renewable raw materials.

If olefins or olefin-containing mixtures are used in the process according to the invention as unsaturated hydrocarbons, the olefins are preferably selected from n-octenes, 1-octene and C₈-containing olefin mixtures.

In the process according to the invention, preferably in a first process step, phosphoramidites of the formula (I):

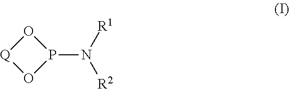

where Q is selected from substituted or unsubstituted 1,1′-biphenyl radicals, R¹ is hydrogen and R² is C₁-C₁₀-alkyl, preferably C₁-C₅-alkyl, or substituted or unsubstituted C₄-C₆-cycloalkyl or phenyl radicals and where R² is not a tert-butyl radical;
preferred substituted 1,1′-biphenyl radicals are those having, in the 3,3′ and/or 5,5′ positions of the 1,1′-biphenyl-2,2′-diol base skeleton, an alkyl radical, preferably a C₁-C₄-alkyl radical and more preferably a tert-butyl radical (t-Bu);
preferred radicals for R² are unbranched and/or branched C₁-C₅-alkyl, substituted or unsubstituted C₄-C₆-cycloalkyl radicals, phenyl radicals excluding tert-butyl (t-Bu);
it is advantageous here when the phosphoramidites of the formula (I) are selected from:

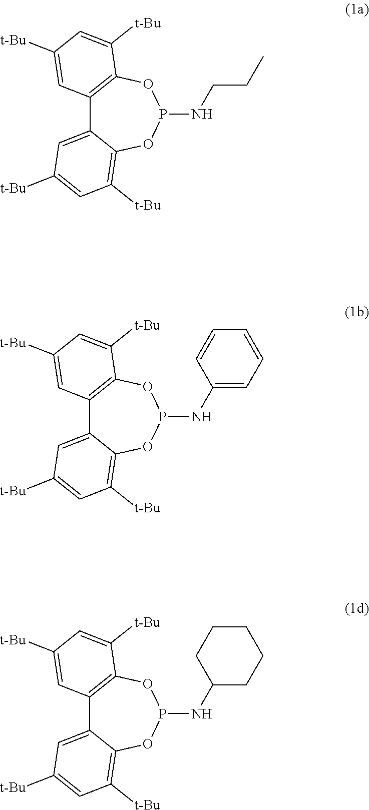 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1]

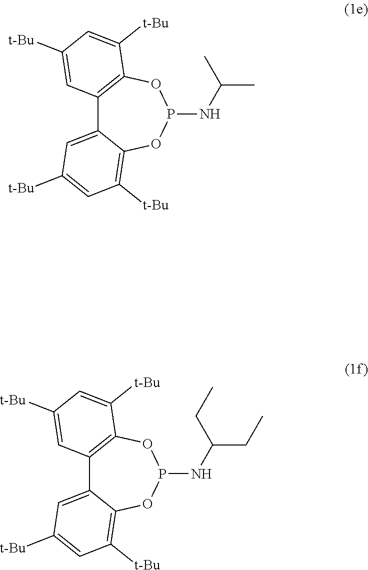 [SMILES: CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

are initially charged as ligands in at least one reaction zone, reacted with a precursor of the transition metal to give a transition metal compound of the formula Me(acac)(CO)L with Me=transition metal and L of the general formula (I):

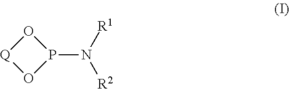

where Q is selected from substituted or unsubstituted 1,1′-biphenyl radicals, R¹ is hydrogen and R² is C₁-C₁₀-alkyl, preferably C₁-C₅-alkyl, or substituted or unsubstituted C₄-C₆-cycloalkyl or phenyl radicals and where R² is not a tert-butyl radical; and where the transition metal Me is selected from ruthenium, cobalt, rhodium and iridium, and is preferably rhodium;
preferred substituted 1,1′-biphenyl radicals are those having, in the 3,3′ and/or 5,5′ positions of the 1,1′-biphenyl-2,2′-diol base skeleton, an alkyl radical, preferably a C₁-C₄-alkyl radical and more preferably a tert-butyl radical (t-Bu).

Preferred radicals for R² are unbranched and/or branched C₁-C₅-alkyl, substituted or unsubstituted C₄-C₆-cycloalkyl radicals, phenyl radicals excluding tert-butyl (t-Bu).

It is advantageous here when L is selected from:

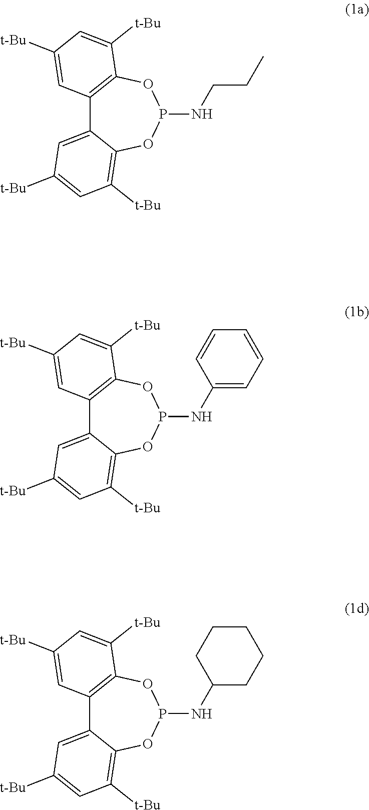 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1]

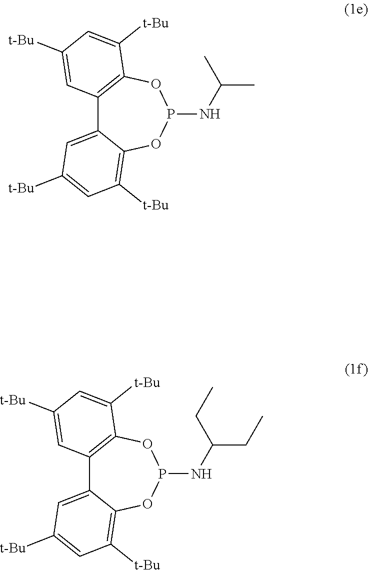 [SMILES: CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

and optional, preferably compulsory, further addition of free ligands of the formula (I):

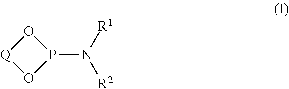

where Q is selected from substituted or unsubstituted 1,1′-biphenyl radicals, R¹ is hydrogen and R² is C₁-C₁₀-alkyl, preferably C₁-C₅-alkyl, or substituted or unsubstituted C₄-C₆-cycloalkyl or phenyl radicals and where R² is not a tert-butyl radical;
it is advantageous here when the ligands of the formula (I) are selected from:

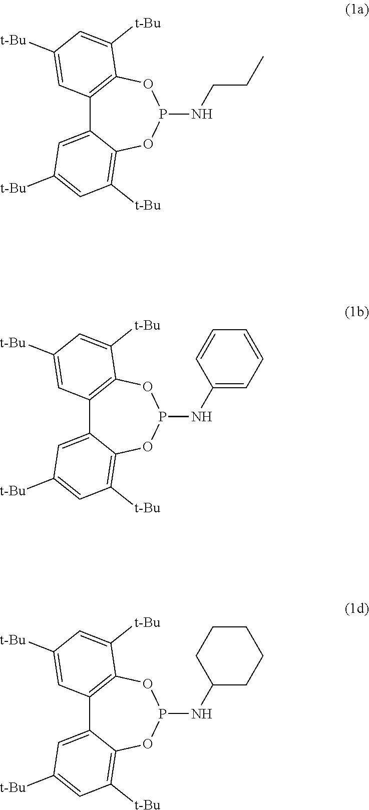 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1]

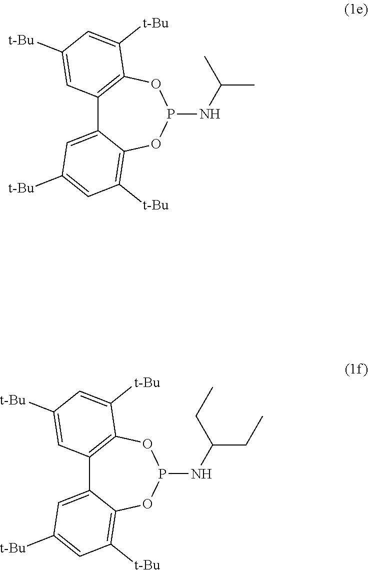 [SMILES: CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

and also solvents and a carbon monoxide- and hydrogen-containing gas mixture, are converted to a catalytically active composition as described above in the hydroformylation;
in a subsequent step, the unsaturated compounds are added under the reaction conditions to form a polyphasic reaction mixture;
after the end of the reaction, the reaction mixture is separated into aldehydes, alcohols, high boilers, ligands and/or, preferably and, degradation products of the catalytically active composition.

In the process according to the invention, the unsaturated compound(s) are preferably added together with the precursor of the transition metal and the ligands (compounds of the formula (I); this is especially preferred when the unsaturated compound(s) are in a liquid state of matter at room temperature and standard pressure corresponding to 1013 hPa.

The hydroformylation is conducted under standard reaction conditions; a temperature of 60° C. to 160° C. and a synthesis gas pressure of 1.0 MPa to 10 MPa are preferred; a temperature of 80° C. to 100° C. and a synthesis gas pressure of 2.0 MPa to 5.0 MPa are especially preferred.

In the context of this invention, degradation products are regarded as being substances which originate from the breakdown of the composition catalytically active in the hydroformylation. They are described, for example, in U.S. Pat. No. 5,364,950, U.S. Pat. No. 5,763,677, and also in Catalyst Separation, Recovery and Recycling, edited by D. J. Cole-Hamilton, R. P. Tooze, 2006, NL, pages 25-26, and in Rhodium-catalyzed Hydroformylation, ed. by P. W. N. M. van Leeuwen and C. Claver, Kluwer Academic Publishers 2006, A A Dordrecht, NL, pages 206-211.

The present invention finally provides a polyphasic reaction mixture comprising:
unsaturated compounds;
a gas mixture including carbon monoxide, hydrogen;
catalytically active compositions comprising:
   a) transition metal compounds of the formula Me(acac)(CO)L with Me=transition metal and L of the general formula (I):

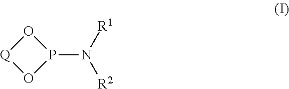

where Q is selected from substituted or unsubstituted 1,1′-biphenyl radicals, R¹ is hydrogen and R² is C₁-C₁₀-alkyl, preferably C₁-C₅-alkyl, substituted or unsubstituted C₄-C₆-cycloalkyl or phenyl radicals, and where R² is not a tert-butyl radical; and where the transition metal Me is selected from ruthenium, cobalt, rhodium and iridium and is preferably rhodium;
preferred substituted 1,1′-biphenyl radicals are those having, in the 3,3′ and/or 5,5′ positions of the 1,1′-biphenyl-2,2′-diol base skeleton, an alkyl radical, preferably a C₁-C₄-alkyl radical and more preferably a tert-butyl radical (t-Bu);
preferred radicals for R² are unbranched and/or branched C₁-C₅-alkyl, substituted or unsubstituted C₄-C₆-cycloalkyl radicals, phenyl radicals excluding tert-butyl (t-Bu);
it is advantageous here when L is selected from:

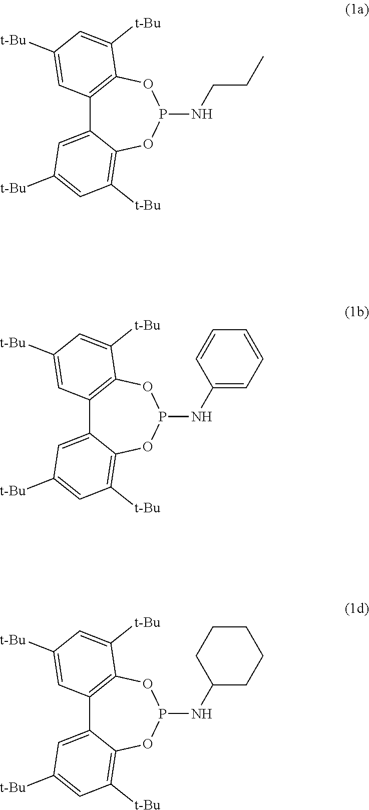 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1]

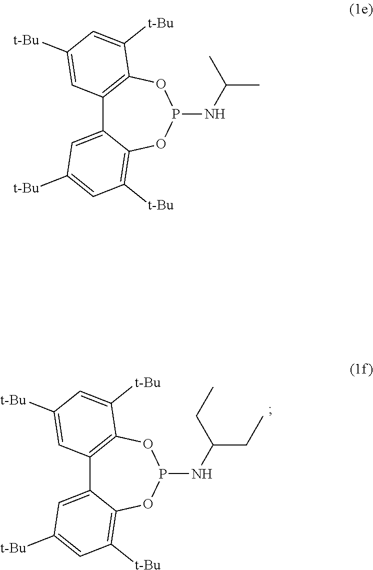 [SMILES: CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

b) free ligands of the formula (I):

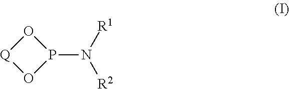

where Q is selected from substituted or unsubstituted 1,1′-biphenyl radicals, R¹ is hydrogen and R² is C₁-C₁₀-alkyl, preferably C₁-C₅-alkyl, substituted or unsubstituted C₄-C₆-cycloalkyl or phenyl radicals, and where R² is not a tert-butyl radical;
preferred substituted 1,1′-biphenyl radicals are those having, in the 3,3′ and/or 5,5′ positions of the 1,1′-biphenyl-2,2′-diol base skeleton, an alkyl radical, preferably a C₁-C₄-alkyl radical and more preferably a tert-butyl radical (t-Bu);
preferred radicals for R² are unbranched and/or branched C₁-C₅-alkyl, substituted or unsubstituted C₄-C₆-cycloalkyl radicals, phenyl radicals excluding tert-butyl (t-Bu);
it is advantageous here when the ligands of the formula (I) are selected from:

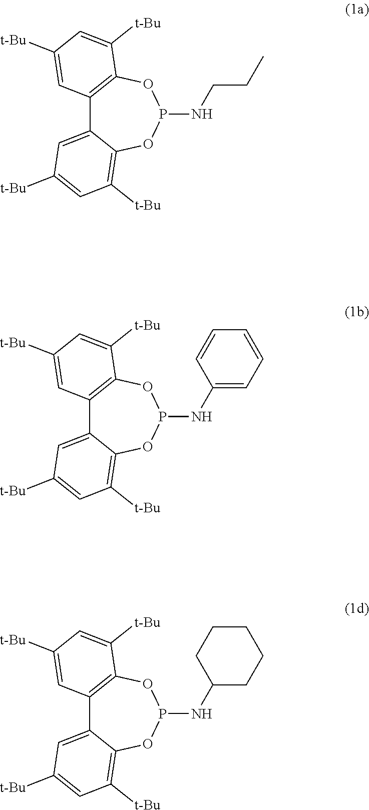 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1]

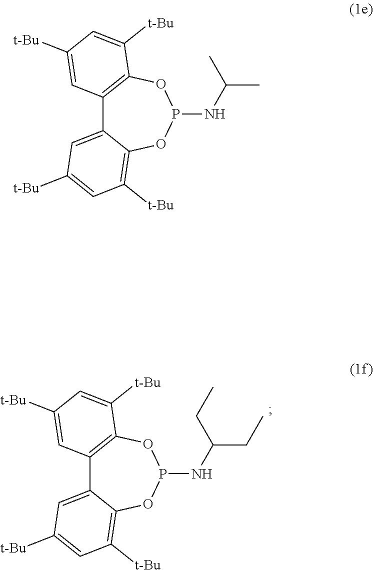 [SMILES: CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

c) solvents.

The process according to the invention is more preferably conducted in such a way that unsaturated compounds are selected from:
hydrocarbon mixtures from steamcracking plants;
hydrocarbon mixtures from catalytically operated cracking plants, for example FCC cracking plants;
hydrocarbon mixtures from oligomerization processes in the homogeneous phase and heterogeneous phases, for example the OCTOL, DIMERSOL, Fischer-Tropsch, Polygas, CatPoly, InAlk, Polynaphtha, Selectopol, MOGD, COD, EMOGAS, NExOCTANE or SHOP process;
hydrocarbon mixtures comprising polyunsaturated compounds;
unsaturated carboxylic acid derivatives;
   and where the solvent is added externally and does not intervene in an inhibiting fashion in the hydroformylation reaction, especially when the solvent is formed in situ from the primary products.

### EXAMPLES

### General Working Methods

All the preparations which follow were conducted with standard Schlenk technology under protective gas. The solvents were dried over suitable desiccants before use (Purification of Laboratory Chemicals, W. L. F. Armarego (Author), Christina Chai (Author), Butterworth Heinemann (Elsevier), 6th edition, Oxford 2009).

Phosphorus trichloride (Aldrich) was distilled under argon before use. All preparative operations were effected in baked-out vessels. The products were characterized by means of NMR spectroscopy. Chemical shifts are reported in ppm. The ³¹P NMR signals were referenced according to: SR_{31P}=SR_{1H}*(BF_{31P}/BF_{1H})=SR_{1H}*0.4048. (Robin K. Harris, Edwin D. Becker, Sonia M. Cabral de Menezes, Robin Goodfellow, I, and Pierre Granger, Pure Appl. Chem., 2001, 73, 1795-1818; Robin K. Harris, Edwin D. Becker, Sonia M. Cabral de Menezes, Pierre Granger, Roy E. Hoffman and Kurt W. Zilm, Pure Appl. Chem., 2008, 80, 59-84).

The recording of nuclear resonance spectra was effected on Bruker Avance 300 or Bruker Avance 400, gas chromatography analysis on Agilent GC 7890A, elemental analysis on Leco TruSpec CHNS and Varian ICP-OES 715, and ESI-TOF mass spectrometry on Thermo Electron Finnigan MAT 95-XP and Agilent 6890 N/5973 instruments.

### Example 1

### General Synthesis Method

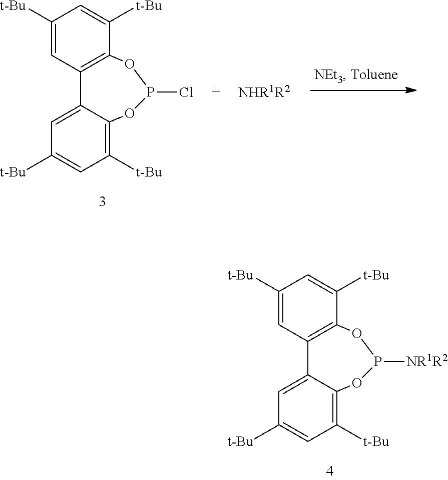

To a stirred solution of the chlorophosphite 3 (4 mmol) (preparation according to US 20080188686 A1) in toluene (15 ml) were added Et₃N (8 mmol) and the appropriate amine (4.8 mmol). The solution was stirred at room temperature. The progress of the reaction was monitored by means of ³¹P NMR spectroscopy. Once the chlorophosphite had been fully converted (2-10 h), the readily evaporable liquids were distilled off under reduced pressure. Subsequently, dried toluene (15 ml) was again added. The resultant suspension was filtered through a layer of neutral alumina (about 3 cm, φ=2 cm; Schlenk filter, porosity 4) and then washed through with toluene (10 ml). After the solution had been concentrated, the residue was dried under reduced pressure at 45-50° C. for 3 h. If necessary, the product can be purified by recrystallization.

### Example 2

### N-(2,4,8,10-Tetra-tert-butyldibenz[d,f]{1,3,2}dioxaphosphepin-6-yl)(n-propyl)amine

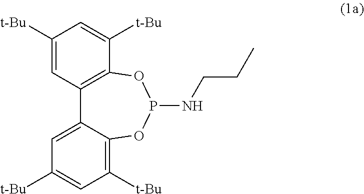 [SMILES: CCCNp1oc2c(C(C)(C)C)cc(C(C)(C)C)cc2c2cc(C(C)(C)C)cc(C(C)(C)C)c2o1]

The compound was prepared analogously to the method of Example 1. Yield: 98%; white solid; ¹H NMR (300 MHz, CDCl₃): δ 0.72 (t, 3H, J=7.4 Hz), 1.26 (s, 18H), 1.36-1.38 (2× overlapping singlets, 20H), 2.67 (pentet, 2H, J=7.4 Hz), 2.84-3.00 (m, 1H), 7.07 (d, 2H, J=2.4 Hz), 7.32 (d, 2H, J=2.4 Hz). ³¹P NMR (121 MHz, CDCl₃): δ 148.0 (s). ¹³C NMR (62 MHz, CDCl₃): δ 11.1 (s, CH₃), 26.0 (d, J=3.4 Hz, CH₂), 31.2 (d, J=2.8 Hz, (CH₃)₃C), 31.6 (s, (CH₃)₃C), 34.6 (s, (CH₃)₃C), 35.6 (s, (CH₃)₃C), 42.4 (d, J=14.0 Hz, CH₂), 124.0 (s, CH_{Ar}), 126.2 (s, CH_{Ar}), 133.1 (d, J=3.5 Hz, C_{Ar}), 140.0 (d, J=1.8 Hz, C_{Ar}), 145.7 (s, C_{Ar}), 147.0 (d, J=5.2 Hz, C_{Ar}). HRMS (EI): calculated m/z (C₃₁H₄₈N₁O₂P₁) 497.34172. Found 497.34214. MS (EI, 70 eV): m/z (1, %): 497 (69), 482 (100), 439 (40), 57 (46). Anal. calculated for C₃₁H₄₈N₁O₂P₁: C, 74.81; H, 9.72; N, 2.81; P, 6.22. Found: C, 73.67; H, 9.65; N, 2.65; P, 6.56.

### Example 3

### N-(2,4,8,10-Tetra-ter-butyldibenz[d,f]{1,3,2}dioxaphosphepin-6-yl)aniline

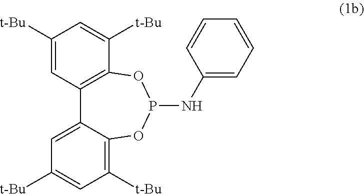 [SMILES: CC(C)(C)c1cc(C(C)(C)C)c2op(Nc3ccccc3)oc3c(C(C)(C)C)cc(C(C)(C)C)cc3c2c1]

The compound was prepared analogously to the method of Example 1. Yield: 35%; white solid (after recrystallizing twice from heptane/toluene (3/2)). ¹H NMR (300 MHz, CDCl₃): δ 1.29 (s, 18H), 1.34 (s, 18H), 5.36 (br, s, 1H), 6.81 (t, 1H, J=7.2 Hz), 6.89 (d, 2H, J=8.0 Hz), 7.07-7.17 (m, 4H), 7.35 (d, 2H, J=2.5 Hz). ³¹P NMR (121 MHz, CDCl₃): δ 140.1 (s). ¹³C NMR (75 MHz, CDCl₃): δ 31.4 (d, J=2.8 Hz, (CH₃)₃C), 31.6 (s, (CH₃)₃C), 34.7 (s, (CH₃)₃C), 35.5 (s, (CH₃)₃C), 117.0 (d, J=13.4 Hz, CH_{Ar}), 120.8 (s, CH_{Ar}), 124.3 (s, CH_{Ar}), 126.3 (s, CH_{Ar}), 129.3 (s, CH_{Ar}), 133.2 (d, J=3.9 Hz, C_{Ar}), 140.5 (d, J=1.9 Hz, C_{Ar}), 142.3 (d, J=17.7 Hz, C_{Ar}), 145.9 (d, J=5.0 Hz, C_{Ar}), 146.4 (s, C_{Ar}). MS (EI, 70 eV): m/z (1, %): 531 (67), 516 (24), 439 (100), 57 (23). HRMS (EI): calculated m/z (C₃₄H₄₆N₁O₂P₁) 531.32607. Found 531.32704. Anal. calculated for C₃₄H₄₆N₁O₂P₁: C, 76.80; H, 8.72; N, 2.63; P, 5.83. Found: C, 76.60; H, 8.88; N, 2.41; P, 5.88.

### Comparative Example 4

### N-(2,4,8,10-Tetra-tert-butyldibenz[d,f]{1,3,2}dioxaphosphepin-6-yl)-tert-butylamine

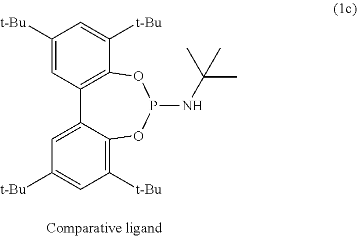 [SMILES: CC(C)(C)Np1oc2c(C(C)(C)C)cc(C(C)(C)C)cc2c2cc(C(C)(C)C)cc(C(C)(C)C)c2o1]

The compound was prepared analogously to the method of Example 1. Yield: 53%; white solid (recrystallized from CH₃CN/toluene (4/1)). ¹H NMR (300 MHz, CDCl₃): δ 1.26-1.27 (2× overlapping signals: s, 18H and d, 9H, J=1.2 Hz), 1.42 (s, 18H), 3.11 (d, 1H, ²J=5.0 Hz), 7.07 (d, 2H, J=2.4 Hz), 7.33 (d, 2H, J=2.4 Hz). ³¹P NMR (121 MHz, CDCl₃): δ 150.4 (s). ¹³C NMR (62 MHz, CDCl₃): δ 31.5-31.6 (2× overlapping singlets, 2×(CH₃)₃CPh), 32.9 (s, (CH₃)₃CNH), 34.6 (s, (CH₃)₃C), 35.4 (s, (CH₃)₃C), 51.3 (d, ²J=17.7 Hz, (CH₃)₃CNH), 124.0 (s, CH_{Ar}), 126.2 (s, CH_{Ar}), 133.3 (d, J=3.7 Hz, C_{Ar}), 140.2 (d, J=1.6 Hz, C_{Ar}), 145.6 (s, C_{Ar}), 146.5 (d, J=6.8 Hz, C_{Ar}). HRMS (ESI-TOF/MS): calculated m/z (C₃₂H₅₁N₁O₂P₁, (M+H)⁺) 512.36519. Found 512.36534; MS (EI, 70 eV): r/z (l, %): 512 (7), 496 (26), 439 (28), 57 (100). Anal. calculated for C₃₂H₅₀N₁O₂P₁: C, 75.11; H, 9.85; N, 2.74; P, 6.05. Found: C, 74.91; H, 9.81; N, 3.00; P, 6.11.

### Example 5

### N-(2,4,8,10-Tetra-tert-butyldibenz[d,f]{1,3,2}dioxaphosphepin-6-yl)cyclohexylamine

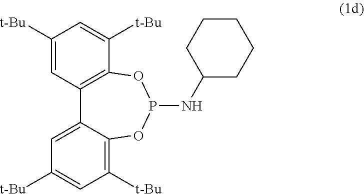 [SMILES: CC(C)(C)c1cc(C(C)(C)C)c2op(NC3CCCCC3)oc3c(C(C)(C)C)cc(C(C)(C)C)cc3c2c1]

The compound was prepared analogously to the method of Example 1. Yield: 92%; white solid. H NMR (300 MHz, CDCl₃): δ 0.99 (m, 5H), 1.27 (s, 18H), 1.41 (s, 18H), 1.48-1.59 (m, 2H), 1.63-1.77 (m, 2H), 2.79-3.03 (m, 2H), 7.07 (d, 2H, J=2.5 Hz), 7.33 (d, 2H, J=2.5 Hz). ³¹P NMR (121 MHz, CDCl₃): δ 150.0 (s). ¹³C NMR (62 MHz, CDCl₃): δ 25.3 (s, CH₂), 25.5 (s, CH₂), 31.4 (d, J=2.8 Hz, (CH₃)₃C), 31.6 (s, (CH₃)₃C, 34.6 (s, (CH₃)₃C), 35.3 (s, (CH₃)₃C), 36.8 (d, J=3.9 Hz, CH₂), 50.6 (d, ²J=14.6 Hz, CH), 123.9 (s, CH_{Ar}), 126.1 (s, CH_{Ar}), 133.0 (d, J=3.7 Hz, C_{Ar}), 140.0 (d, J=1.8 Hz, C_{Ar}), 145.6 (s, C_{Ar}), 146.8 (d, J=6.0 Hz, C_{Ar}). HRMS (ESI-TOF/MS): calculated n/z (C₃₄H₅₃N₁O₂P₁, (M+H)⁺) 538.38084. Found 538.38138; MS (EI, 70 eV): m/z (1, %): 537 (96), 522 (36), 440 (75), 57 (40). Anal. calculated for C₃₄H₅₂N₁O₂P₁: C, 75.94; H, 9.75; N, 2.60; P, 5.76. Found: C, 75.57; H, 9.67; N, 2.78; P, 5.81.

### Example 6

### N-(2,4,8,10-Tetra-tert-butyldibenz[d,f]{1,3,2}dioxaphosphepin-6-yl)isopropylamine

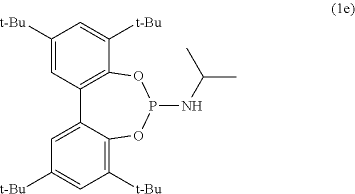 [SMILES: CC(C)Np1oc2c(C(C)(C)C)cc(C(C)(C)C)cc2c2cc(C(C)(C)C)cc(C(C)(C)C)c2o1]

The compound was prepared analogously to the method of Example 1. Yield: 93%; white solid. ¹H NMR (250 MHz, CDCl₃): δ 0.99 (d, 6H, J=6.3 Hz), 1.27 (s, 18H), 1.41 (s, 18H), 2.73-2.87 (m, 1H), 3.28-3.48 (m, 1H), 7.07 (d, 2H, J=2.4 Hz), 7.33 (d, 2H, J=2.4 Hz). ³¹P NMR (101 MHz, CDCl₃): δ 150.0 (s). ¹³C NMR (62 MHz, CDCl₃): δ 26.3 (d, J=4.4 Hz, CH₃), 31.4 (d, J=2.8 Hz, (CH₃)₃C), 31.6 (s, (CH₃)₃C), 34.6 (s, (CH₃)₃C), 35.3 (s, (CH₃)₃C), 43.7 (d, ²J=18.5 Hz, CH), 124.0 (s, CH_{Ar}), 126.1 (s, CH_{Ar}), 133.1 (d, J=3.7 Hz, C_{Ar}), 140.0 (d, J=1.9 Hz, C_{Ar}), 145.7 (s, C_{Ar}), 146.8 (d, J=5.7 Hz, C_{Ar}). HRMS (ESI-TOF/MS): calculated m/z (C₃₁H₄₉N₁O₂P₁, (M+H)⁺) 498.34954. Found 498.3497; MS (EI, 70 eV): m/z (l, %): 497 (100), 482 (99), 439 (37), 57 (43). Anal. calculated for C₃₁H₄₈N₁O₂P₁: C, 74.81; H, 9.72; N, 2.81; P, 6.22. Found: C, 74.80; H, 9.89; N, 2.63; P, 6.40.

### Example 7

### N-(2,4,8,10-Tetra-tert-butyldibenz[d,f]{1,3,2}dioxaphosphepin-6-yl)-3-aminopentane

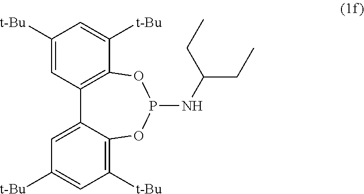 [SMILES: CCC(CC)Np1oc2c(C(C)(C)C)cc(C(C)(C)C)cc2c2cc(C(C)(C)C)cc(C(C)(C)C)c2o1]

The compound was prepared analogously to the method of Example 1. Yield: 75%; white solid. ¹H NMR (300 MHz, CDCl₃): δ 0.77 (t, 6H, J=7.5 Hz), 1.27 (s, 18H), 1.29-1.39 (m, 4H), 1.41 (s, 18H), 2.88 (t, 1H, J=8.4 Hz), 3.00-3.15 (m, 1H), 7.07 (d, 2H, J=2.5 Hz), 7.33 (d, 2H, J=2.5 Hz). ³¹P NMR (121 MHz, CDCl₃): L 149.7 (s). ¹³C NMR (62 MHz, CDCl₃): δ 9.8 (s, CH₃CH₂), 28.6 (d, ³J=4.6 Hz, CH₃CH₂), 31.3 (d, J=2.9 Hz, (CH₃)₃C), 31.6 (s, (CH₃)₃C), 34.6 (s, (CH₃)₃C), 35.4 (s, (CH₃)₃C), 54.2 (d, ²J=19.6 Hz, CH), 124.0 (s, CH_{Ar}). 126.3 (s, CH_{Ar}), 133.1 (d, J=3.7 Hz, C_{Ar}), 140.0 (d, J=1.8 Hz, C_{Ar}), 145.6 (s, C_{Ar}), 146.8 (d, J=6.5 Hz, C_{Ar}). HRMS (ESI-TOF/MS): calculated m/z (C₃₃H₅₃N₁O₂P₁, (M+H)⁺) 526.38084. Found 526.38131; MS (EI, 70 eV): m/z (l, %): 525 (34), 496 (84), 439 (100), 57 (25). Anal. calculated for C₃₃H₅₂N₁O₂P₁: C, 75.39; H, 9.97; N, 2.66; P, 5.89. Found: C, 75.30; H, 9.72; N, 2.28; P, 5.85.

### Example 8

### General Method for the Synthesis of Rh(Acac)(CO)L from the Transition Metal Precursor

To a stirred solution of Rh(acac)(CO)₂ (1 mmol) in dried CH₂Cl₂ (8 ml) was added dropwise, within 40 min, a solution of the phosphoramidite (1 mmol) in dried CH₂Cl₂ (8 ml). The solution was stirred at room temperature for 2 h. Subsequently, the solvent was distilled off under reduced pressure and the residue was dried in vacuo for 1 h.

### Example 9

### Rh-Containing Complex with Ligand (1c)

The compound was synthesized analogously to the method detailed in Example 8. Yield: 98%; green powder. ¹H NMR (250 MHz, CDCl₃): δ 0.97 (s, 9H), 1.25 (s, 18H), 1.55 (s, 18H), 1.91 (s, 3H), 1.97 (s, 3H), 4.53 (d, 2H, J=23.7 Hz, 1H), 5.40 (s, 1H), 7.06 (d, 2H, J=2.4 Hz), 7.39 (d, 2H, J=2.5 Hz). ³¹P NMR (101 MHz, CDCl₃): δ 134.34 (d, ¹J_{RhP}=268.3 Hz). ¹³C NMR (75 MHz, CDCl₃): δ 27.5 (s, CH_{3acac}), 27.7 (d, J=7.0 Hz, CH_{3cac}), 31.5-31.6 (overlapping singlets, (CH₃)₃C and (CH₃)₃CN), 32.3 (s, (CH₃)₃C), 34.6 (s, (CH₃)₃C), 35.6 (s, (CH₃)₃C), 100.8 (s, CH_{acac}), 124.7 (s, CH_{Ar}), 126.5 (s, CH_{Ar}), 131.8 (d, J=2.6 Hz, C_{Ar}), 139.8 (d, J=3.4 Hz, C_{Ar}), 146.3 (d, J=12.3 Hz, C_{Ar}), 146.6 (d, J=1.8 Hz, C_{Ar}), 184.8 (s, CH₃CO_{acac}), 188.7 (s, CH₃CO_{acac}). MS (EI, 70 eV): m/z (l, %): 741 (37), 713 (100), 613 (19), 578 (36), 557 (67), 439 (12), 57 (18). HRMS (ESI-TOF/MS): calculated m/z (C₃₈H₅₈N₁O₅P₁Rh₁, (M+H)⁺) 742.31022. Found 742.31125; calculated m/z (C₃₈H₅₇N₁Na₁O₅P₁Rh₁Na, (M+Na)⁺) 764.29216. Found 764.29301. Anal. calculated for C₃₈H₅₇N₁O₅P₁Rh₁: C, 61.53; H, 7.75; N, 1.89; P, 4.18; Rh, 13.87. Found: C, 61.24; H, 7.66; N, 1.84; P, 4.36; Rh, 13.93. IR (CaF₂ cuvette 0.1 mm, 0.1 M solution in toluene): 2000 cm⁻¹ (CO).

### Example 10

In the process according to the invention, the hydroformylation was preferably conducted in a 200 ml autoclave equipped with pressure-retaining valve, gas flow meter, sparging stirrer and pressure pipette as reaction zone. To minimize the influence of moisture and oxygen, the toluene used as solvent was treated with sodium ketyl and distilled under argon. The mixture of the n-octenes used as substrate was heated at reflux over sodium and distilled under argon for several hours. The transition metal was used as a precursor in the form of [(acac)Rh(COD)](acac=acetylacetonate anion; COD=1,5-cyclooctadiene), dissolved in toluene. The latter was mixed with a solution of the respective ligand in the autoclave under an argon atmosphere. The reactor was heated up under synthesis gas pressure and the unsaturated compounds, especially the olefin, the mixture of olefins, were introduced by means of a pressure-resistant pipette once the reaction temperature had been attained. In this case it is advantageous in the process according to the invention, to introduce the unsaturated compounds to be hydroformylated into the reaction zone prior to the addition of the hydrogen- and carbon monoxide-containing gas mixture. This applies especially to unsaturated compounds present in a liquid state at room temperature and standard pressure. In these cases, there is no need to add an external solvent, the solvents being the secondary products formed internally, for example those formed in situ during the reaction from the aldol condensation of the primary aldehyde products.

The reaction was conducted at constant pressure. After the reaction time had elapsed, the autoclave was cooled to room temperature, decompressed while stirring and purged with argon. 1 ml of each reaction mixture was removed immediately after the stirrer had been switched off, diluted with 5 ml of pentane and analysed by gas chromatography. Inventive working examples are compiled in Table 1, in which one entry also relates to the use of the phosphite ligands known by the CAS Registry Numbers [93347-72-9], [31570-04-4]—trade name Alkanox®240.

TABLE 1
Hydroformylation of a mixture of n-octenes^{a,b}

| | | | Yield |
|---|---|---|---|
| Ligand | Formula | k_{obs.}[min⁻¹] | [%] |
| (1a) | 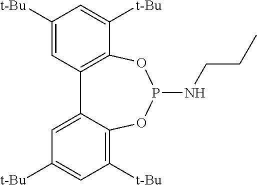 [SMILES: CCCNp1oc2c(C(C)(C)C)cc(C(C)(C)C)cc2c2cc(C(C)(C)C)cc(C(C)(C)C)c2o1] | 0.223 | 100 |
| (1e) | 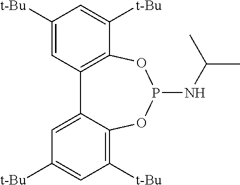 [SMILES: CC(C)Np1oc2c(C(C)(C)C)cc(C(C)(C)C)cc2c2cc(C(C)(C)C)cc(C(C)(C)C)c2o1] | 0.267 | 98 |
| (1f) | 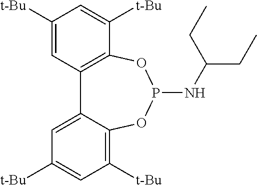 [SMILES: CCC(CC)Np1oc2c(C(C)(C)C)cc(C(C)(C)C)cc2c2cc(C(C)(C)C)cc(C(C)(C)C)c2o1] | 0.302 | 98 |
| (1d) | 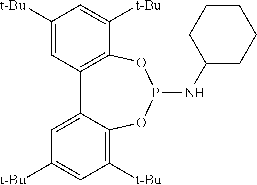 [SMILES: CC(C)(C)c1cc(C(C)(C)C)c2op(NC3CCCCC3)oc3c(C(C)(C)C)cc(C(C)(C)C)cc3c2c1] | 0.252 | 98 |
| Compar- ative ligand (1c) | 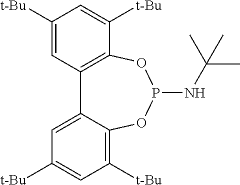 [SMILES: CC(C)(C)Np1oc2c(C(C)(C)C)cc(C(C)(C)C)cc2c2cc(C(C)(C)C)cc(C(C)(C)C)c2o1] | 0.223 | 98 |
| (1b) | 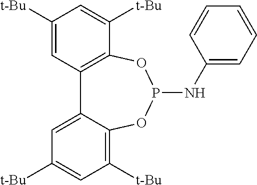 [SMILES: CC(C)(C)c1cc(C(C)(C)C)c2op(Nc3ccccc3)oc3c(C(C)(C)C)cc(C(C)(C)C)cc3c2c1] | 0.230 | 98 |
| Compar- ative ligand | Alkanox ®240 as per CAS Reg. No. [93347- 72-9], [31570-04-4] | 0.194 | 95 |

^{a}conditions: [Rh] = 0.01728 mmol; 40 ppm Rh; P/Rh = 5:1, 5.0 MPa CO/H₂, [1-octene] = about 94 mmol; toluene, 100° C.; 40 min.
^{b}consisting of: 1-octene, 3%; cis+trans-2-octene, 49%; cis+trans-3-octene, 29%; cis+trans-4-octene, 16%; structurally isomeric octenes, 3%.

The relative activities are determined by the ratio of 1st order k to k0, i.e. the k value at time 0 in the reaction (start of reaction), and describe the relative decrease in activity during the experiment duration.

The 1st order k values are obtained from a plot of (−ln(1-conversion)) against time.

The results of the hydroformylations disclosed in Table 1 show that the ligands according to the invention have improved yields of aldehydes and, in the case of ligands (1b), (1d), (1e) and (1f) according to the invention, a distinctly improved catalytic efficacy, measured as activity k_{obs}. [min⁻¹], compared to the comparative ligands (1c) and Alkanox®240.

The enhanced activity compared to the comparative ligands is of particular relevance for use in industrial scale processes, since a higher activity means shorter residence times for target products in the reaction zone and hence the overall economic viability of the industrial scale process is optimized.

Even in the case of an equal catalytic efficacy, measured as activity k_{obs}. [min⁻¹], of the ligand (1a) according to the invention with the comparative ligand (1c), it remains to be emphasized that the ligand (1a) according to the invention generates a better yield of product than the comparative ligand (1c); see Table 1.

In addition, in the reaction, a maximum phosphorus/rhodium ratio—referred to as P/Rh for short or else ligand/transition metal ratio—of 5:1 was established. Thus, it is necessary to use much less ligand in the hydroformylation step compared to the prior art (A. van Rooy, D. Burgers, P. C. J. Kamer, P. W. N. M. van Leeuwen, Rec. Tray. Chim. Pays-Bas 1996, 115, 492). This is of particular relevance since the ligands can make up a large portion of the process costs. Thus, if less ligand is required, this has an immediate positive effect on the overall economic viability of the industrial scale process.

It has been found that the object stated at the outset of providing ligands for catalytically active compositions having a maximum or improved catalytic activity compared to the ligands already described in the prior art is achieved by the ligands according to the invention—(1a), (1b), (1d), (1e) and (1f).

### Example 11

### Hydroformylation of Methyl Oleate

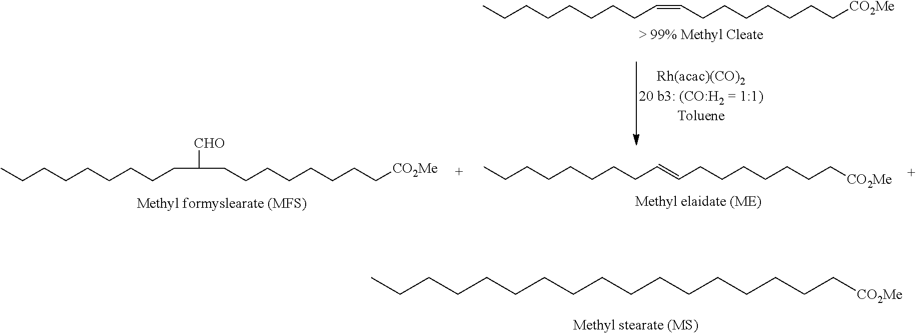 [SMILES: CCCCCCCC/C=C\CCCCCCCC(=O)OC ; CCCCCCCCCC(C=O)CCCCCCCCC(=O)OC ; CCCCCCCC/C=C/CCCCCCCC(=O)OC ; CCCCCCCCCCCCCCCCCC(=O)OC]

### Abbreviations Used:

MO=methyl oleate
MFS=methyl formylstearate
MS=methyl stearate
ME=methyl elaidate

| | | T | t | Conversion | MFS | ME |
|---|---|---|---|---|---|---|
| Experiment^{a} | Ligand | [° C.] | [h] | [%]^{b} | [%]^{c} | [%]^{c} |
| 1 | (1a) | 80 | 6 | 92.9 | 43.8 | 49.0 |

^{a}The reaction was conducted with 1.0 mmol of substrate in 10 ml of toluene, a ratio of substrate:Rh = 910:1, ligand:Rh = 25:1 over 6 h; the reaction mixture at 2.0 MPa CO/H₂ was heated to 80° C. over 6 h and then the stirring was continued at room temperature under synthesis gas pressure until workup.
^{b}The conversion was determined by gas chromatography and calculated as follows: 100-% GC yield of MO. These values are an approximation, since the gas chromatography separation of MO and ME is incomplete.
^{c}Yield determined by gas chromatography. In all cases, the formation of the hydrogenated saturated methyl stearate product (MS) was less than 0.1%.

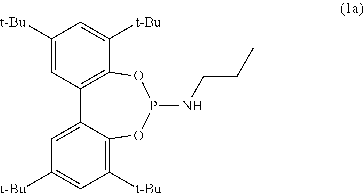 [SMILES: CCCNp1oc2c(C(C)(C)C)cc(C(C)(C)C)cc2c2cc(C(C)(C)C)cc(C(C)(C)C)c2o1]

The inventive ligand (1a) shows a high yield in the conversion to the methyl formylstearate product MFS, and also in the isomerization to give the trans isomer of the methyl oleate substrate, which is referred to as methyl elaidate ME, and only a low hydrogenation activity is registered, as already stated above, the phosphorus/rhodium ratio being limited to a maximum of 25:1.

1. Phosphoramidites of the formula (I) 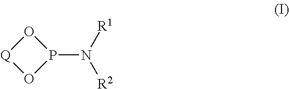
where Q is selected from substituted or unsubstituted 1,1′-biphenyl radicals, R¹ is hydrogen and R² is C₁-C₁₀-alkyl or substituted or unsubstituted C₄-C₆-cycloalkyl or phenyl radicals and where R² is not a tert-butyl radical.

2. Phosphoramidites according to claim 1 selected from:

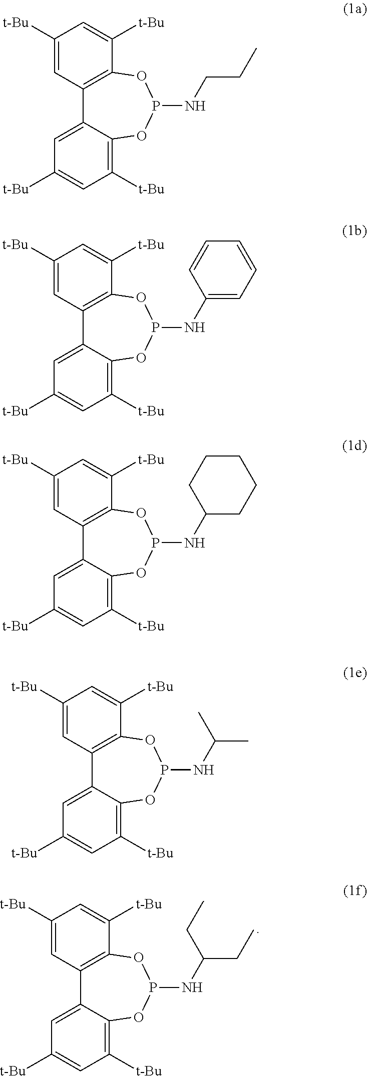 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1 ; CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

3. Transition metal compounds of the formula Me(acac)(CO)L with Me=transition metal and L is of the general formula (I): 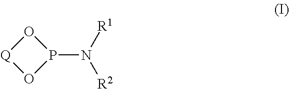
where Q is selected from substituted or unsubstituted 1,1′-biphenyl radicals, R¹ is hydrogen and R² is C₁-C₁₀-alkyl or substituted or unsubstituted C₄-C₆-cycloalkyl or phenyl radicals, R² is not tert-butyl, and where the transition metal Me is selected from ruthenium, cobalt, rhodium and iridium.

4. Compound according to claim 3, where L is selected from:

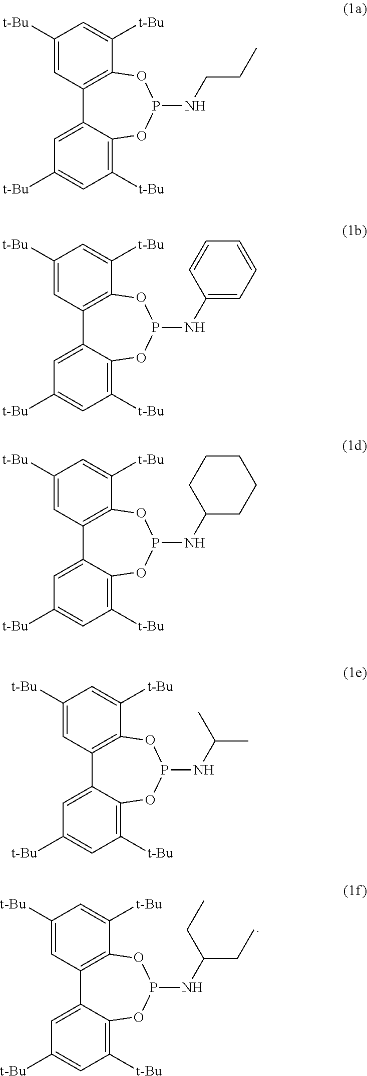 [SMILES: CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(Nc3ccccc3)oc3c(C(C)(C)C)cc(C(C)(C)C)cc3c2c1 ; CC(C)(C)c1cc(C(C)(C)C)c2op(NC3CCCCC3)oc3c(C(C)(C)C)cc(C(C)(C)C)cc3c2c1 ; CC(C)Np1oc2c(C(C)(C)C)cc(C(C)(C)C)cc2c2cc(C(C)(C)C)cc(C(C)(C)C)c2o1 ; CCC(CC)Np1oc2c(C(C)(C)C)cc(C(C)(C)C)cc2c2cc(C(C)(C)C)cc(C(C)(C)C)c2o1]

5. Compound according to claim 3, where the transition metal is rhodium.

6. Catalytically active compositions in the hydroformylation comprising:
a) transition metal compounds according to claim 3;
b) optionally free ligands of the formula (I) 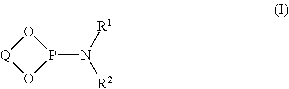
where Q is selected from substituted or unsubstituted 1,1′-biphenyl radicals, R¹ is hydrogen and R² is C₁-C₁₀-alkyl or substituted or unsubstituted C₄-C₆-cycloalkyl or phenyl radicals and where R² is not a tert-butyl radical;
c) solvents.

7. A process for hydroformylating unsaturated compounds comprising introducing the catalytically active composition according to claim 6.

8. Process for hydroformylating unsaturated compounds using a catalytically active composition according to claim 6, where the unsaturated compounds are selected from:
hydrocarbon mixtures from steamcracking plants;
hydrocarbon mixtures from catalytically operated cracking plants;
hydrocarbon mixtures from oligomerization processes;
hydrocarbon mixtures comprising polyunsaturated compounds;
olefin-containing mixtures including olefins having up to 30 carbon atoms;
unsaturated carboxylic acid derivatives.

9. Process according to claim 8, wherein, in a first process step, phosphoramidites of the formula (I) 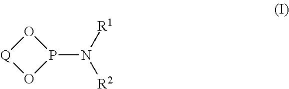
where Q is selected from substituted or unsubstituted, 1,1′-biphenyl radicals, R¹ is hydrogen and R² is C₁-C₁₀-alkyl or substituted or unsubstituted C₄-C₁₀-cycloalkyl or phenyl radicals and where R² is not a tert-butyl radical, are initially charged in at least one reaction zone, and mixed with a precursor of a transition metal to give a transition metal compound of formula Me(acac)(CO)L with Me=transition metal and L is of the general formula (I): 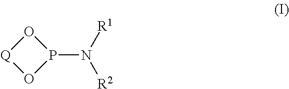
where Q is selected from substituted or unsubstituted 1,1′-biphenyl radicals, R¹ is hydrogen and R² is C₁-C₁₀-alkyl or substituted or unsubstituted C₄-C₆-cycloalkyl or phenyl radicals, R² s not tert-butyl, and where the transition metal Me is selected from ruthenium, cobalt, rhodium and iridium, optionally further phosphoramidites of the formula (I) are added, and also solvents and a carbon monoxide- and hydrogen-containing gas mixture, to give a catalytically active composition comprising a) the transition metal compound, b) optionally free ligands of the formula (I) 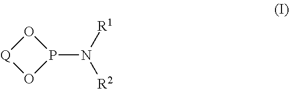
where Q is selected from substituted or unsubstituted 1,1′-biphenyl radicals, R¹ is hydrogen and R² is C₁-C₁₀-alkyl or substituted or unsubstituted C₄-C₆-cycloalkyl or phenyl radicals and where R² is not a tert-buty radical, and c) solvents;
in a subsequent step, the unsaturated compounds are added under the reaction conditions to form a polyphasic reaction mixture;
and after the end of the reaction, the reaction mixture is separated into aldehydes, alcohols, high boilers, ligands and/or degradation products of the catalytically active composition.

10. Polyphasic reaction mixture comprising:
unsaturated compounds,
a gas mixture including carbon monoxide and hydrogen;
aldehydes, and
at least one catalytically active composition according to claim 6.

11. Compound according to claim 4, where the transition metal is rhodium.
